(19) United States
(12) Patent Application Publication
(10) Pub. No.: US 2008/0004458 A1
(43) Pub. Date: Jan. 3, 2008

(54) TRANSESTERIFICATION CATALYST MIXING SYSTEM

(76) Inventors: Rudolf A. Wiedemann, Reno, NV (US); James P. Sheridan, Reno, NV (US); Russell E. Bouydston, Reno, NV (US)

Correspondence Address: IAN F. BURNS & ASSOCIATES, P.O. BOX 71115, RENO, NV, 89570, US

(21) Appl. No.: 11/765,659

(22) Filed: Jun. 20, 2007

Related U.S. Application Data
(60) Provisional application No. 60/805,332, filed on Jun. 20, 2006.

(51) Int. Cl. B01J 8/02; C07C 51/43

(52) U.S. Cl. 554/174; 422/211

(57) ABSTRACT

A transesterification reaction system having a catalyst dissolution chamber, a catalyst-triglyceride mixing chamber, a holding chamber and a fluid transfer device for circulating the triglyceride component, is disclosed. The catalyst dissolution chamber includes a turbulating device for wetting solid catalyst with an alcohol component and the catalyst-triglyceride mixing chamber includes a turbulent flow suction device. A transesterification method using the reaction system described above is also disclosed.

### CROSS REFERNCES TO RELATED APPLICATIONS

This application claims priority to U.S. provisional patent application Ser. No. 60/805,332, filled on Jun. 20, 2006.

### BACKGROUND OF THE INVENTION

The present invention relates to a transesterification catalyst system and a method of use. More specifically, the catalyst system includes an apparatus configured to dissolve the transesterfication catalyst in a premix solution prior to contact with a triglyceride raw material.

Vegetable oils, fats, greases and other sources of triglycerides have been used as a source of ester-based oxygenated fuels. The latter are referred to as “biodiesel” and provide nontoxic, biodegradable alternatives to traditional petroleum diesel fuel. Mixtures or blends of various biodiesel fuels with traditional petroleum diesel fuel can minimize the toxicity and level of diesel exhaust fumes. These mixtures, therefore, provide a source of substitute fuel for standard diesel fuel.

Typically, biodiesel fuels are produced by an acid or base catalyst transesterification reaction of selected raw materials. For example, fats or oils (triglycerides) are contacted with an alcohol in the presence of catalyst to produce glycerol and the corresponding alkyl esters (for example, methyl ester if methanol is used or ethyl ester if ethanol is used); the resultant alkyl ester provide the biodiesel fuel.

The alcohol component is typically used in excess stoichiometric ratio relative to the triglyceride component in order t force the transesterfication reaction to completion, the alcohol component may be recovered for reuse. Typical catalyst include sodium or potassium hydroxide, which may be mixed with the alcohol component prior to contact with the triglyceride component. As the alkyl ester product is produced, it is separated from the glycerin (glycerol) byproduct.

In a typical transesterification reaction, the catalyst (commonly granular solid) is first dissolved in the alcohol component to form a premix solution, and the latter is subsequently mixed with the triglyceride component. The premix solution typically requires use of a separate vessel in order to completely dissolve the catalyst before contact with the triglyceride. Preparation of the catalyst premix solution is an exothermic process and may result in vaporization of the alcohol, pressurization of the premix vessel, subsequent flammability concerns and other safety issues.

The present invention provides a method and apparatus for enhancing the rate of the transesterfication reaction and minimizing flammability and safety issues by mixing a solid catalyst with the alcohol component, followed by enhanced mixing of the catalyst solution and triglyceride components via a turbulent flow suction device.

### BRIEF SUMMARY OF THE INVENTION

The present invention provides a transesterfication reaction apparatus including: (A) a catalyst dissolution chamber comprising (i) catalyst mixing chamber comprising (a) inlets for solid catalyst and alcohol components; and (b) turbulating device for wetting the solid catalyst with alcohol; and (ii) catalyst solution chamber coupled to the catalyst mixing chamber comprising: (a) filtration device located at the interface of the catalyst mixing chamber and the catalyst solution chamber for preventing transmission of solid catalyst from the catalyst mixing chamber to the catalyst solution chamber; and (b) outlet fir catalyst solution; (B) a catalyst-triglyceride mixing chamber comprising (i) turbulent flow suction device, (II) first inlet for transferring the catalyst solution into the turbulent flow suction device, wherein the first inlet is coupled to the outlet from the catalyst solution chamber of the catalyst dissolution chamber; (iii) second inlet for transferring a triglyceride fees lime; and (iv) outlet for transfer of a mixture of the catalyst solution and the triglyceride component and transesterification reaction products; and (D) a fluid transfer device coupled to the holding chamber for transfer of the triglyceride component through the triglyceride feed line to the second inlet of the catalyst-triglyceride mixing chamber.

The turbulating device for wetting the solid catalyst may comprise a nozzle configured to deliver the alcohol tangentially to a wall of the catalyst mixing chamber to create a circular mixing flow and turbulating action with the solid catalyst. In this way the solid catalyst, typically provided in a granular form, may be wetted, mixed and efficiently dissolved into the alcohol while mininizing heat creation.

The filtration device of the catalyst solution chamber typically comprises a metal screen having appropriate mesh size to minimize and prevent any of the undissolved solid catalyst granules form leaving the catalyst mixing chamber before dissolution. Other suitable filtration devices for use in the present invention may include, for example, sieve, strainer and sifting devices.

Turbulent flow suction devices suitable for use in the present invention include, for example, venturi tubes, static mixers and dynamic mixers (such as blending mixer devices). Typically, the turbulent flow suction device used in the catalyst-triglyceride mixing chamber is a venturi tube. The venturi tube may be of various dimensions so as to provide proper suction, flow rates and mixing so that turbulent flow is achieved in the catalyst-triglyceride mixing zone. The catalyst solution (from the catalyst solution chamber) is drawn off from the catalyst dissolution chamber by means of the turbulent flow suction device (for example, venturi tube). One example of a venturi device useful in the present invention is a Model 878-02 Injector, manufactured by Mozzel Injector Corporation, 500 Rooster Dr., Bakersfield, Calif., (see FIG. 2).

Typically, the method of the present invention includes combing the catalyst solution with the triglyceride component in the catalyst-triglyceride mixing chamber in a ratio of from about 1/3 to about 1/10 by volume by adjusting relative flow rates of the catalyst solution and triglyceride component into the first and second inlets, respectively, of the turbulent flow suction device. Representative ratios of catalyst solution to triglyceride components are form about 1/4 to 1/6 by volume; more typically, the ratio is 1 part by volume catalyst solution (premix) to 5 parts triglyceride. This ratio and other ratios of catalyst-triglyceride may be achieved by selection of different venturi dimensions, for example. Typically, the outlet of the catalyst-triglyceride mixing chamber is directed downwards and tangentially into the holding chamber in order to provide homogeneous mixing of the reactant components.

The method of the present invention includes using solid catalyst selected from one of more of the group consisting of lithium hydroxide, sodium hydroxide and potassium hydroxide. Alcohols useful in the transesterification reaction of the present invention include those selected from one or more of the group consisting of methanol, ethanol, n-propanol and isopropanol. triglyceride components useful in the transesterification reaction of the present invention include triglycerides based ester (glycerol) of (C₈-C₂₄)fatty acids. Typical fatty acids includes, for example, caprylic, lauric, myristic, plamitic, stearic, arachnic, behenic, lignoceric, oleic, linoleic, linolenic and erucid acids; more typically, the triglycerides are based on esters of (C₁₆-C₂₀)fatty acids. Sources of the triglyceride components may include, for example, vegetable oils and used cooking oils. Typically, triglyceride materials useful in the present invention contain minimal amounts of water (moisture), that is less than about 0.5 percent by weight. In addition, it is desirable that the triglyceride materials have a minimum of acid contaminants, for example, less than about 3 percent by weight of free fatty acids, preferably less than about 2 percent free fatty acid.

The reaction products (alkyl fatty acid ester, glycerol), along with residual catalyst, residual triglyceride, and excess alcohol are allowed to settle in the holding chamber. The layers separate and the alkyl fatty acid ester (biodiesel) may be separated from the remaining mixture. Washing, purification and recovery of glycerol foe reuse may be carried out by conventional means.

In one embodiment, the reaction apparatus of the present invention includes an arrangement where the container of the holding chamber encloses the catalyst dissolution chamber and the catalyst-triglyceride mixing chamber in an upper portion of the holding chamber, thus allowing for the entire apparatus to be housed in a single unit. Alternatively, the catalyst dissolution chamber (and its component parts) may be suited separately from, and outside of the holding chamber.

Among the advantages of the present invention are those directed to (a) minimizing heat evolution during preparation of the catalyst solution by thoroughly wetting the solid catalyst with alcohol component in the catalyst mixing chamber, (b) minimizing alcohol vaporization and thereby reducing flammability and pressurization potential in the reactor system, (c) enhancing the speed and extent of the transesterification reaction by use of a turbulent flow suction device for mixing the reaction components, and (d) providing a reactor system in one compact container.

The above description sets forth certain features of representative embodiments disclosed herein. There are other features that will become apparent to those skilled in the art from this specification. In this respect, before explaining specific embodiments of the invention in detail, it is to be understood that the invention is not limited in its application to the details of the construction and to the arrangement of the components set forth in the following description or as illustrated in the drawings, nor is the invention necessarily a solution of each problem noted in the background Section above. In addition, the various disclosed embodiments are capable of being practiced and carried out in various ways. Also, it is to be understood that the phraseology and terminology employed herein are for the purpose of brief description and should not be regarded as limiting.

### BRIEF DESCRIPTION OF THE DRAWINGS

FIG. 1A is substantially a cross sectional view of an apparatus of the present invention.

FIG. 1B is substantially a side view of one embodiment of the catalyst mixing chamber of the apparatus.

FIG. 2 is substantially a side view of one embodiment of a turbulent flow suction device useful in the present invention.

### DETAILED DESCRIPTION OF THE INVENTION

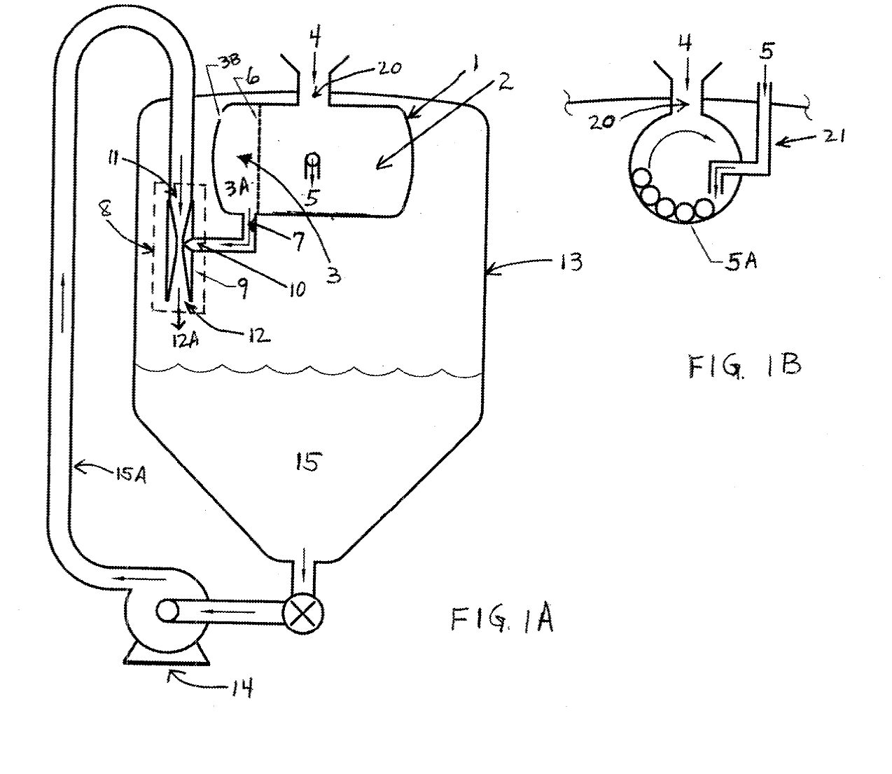

FIG. 1A presents a cross sectional view of an apparatus of the present invention. The transesterification reaction apparatus includes a catalyst dissolution chamber 1 comprising a catalyst mixing chamber 2 and a catalyst solution chamber 3. Catalyst solution chamber 3 may optionally include a vent 3B. A catalyst 4 id added to the catalyst mixing chamber 2 via an inlet 20 (see also FIG. 1B) and an alcohol 5 is introduced via an inlet 21 (see FIG. 1B). The catalyst dissolution chamber 1 includes a filtration device 6 and outlet 7.

A catalyst-triglyceride mixing chamber 8 includes a turbulent flow suction device 9, a first inlet 10 for transferring a catalyst solution 3A into the turbulent flow suction device 9, and a second inlet 11 for transferring a triglyceride component 15 into the turbulent flow device 9. Second inlet 11 is coupled to a triglyceride feed line 15A. An outlet 12 is provided for transfer of the mixture 12A of catalyst solution 3A and triglyceride component 15 into a holding chamber 13. Holding chamber 13 includes a container for holding the triglyceride component 15 and transesterification reaction products. A fluid transfer device 14 (for example, a pump) enables transfer of triglyceride component 15 through the triglyceride feed line 15A to the second inlet 11 of the catalyst-triglyceride mixing chamber 8.

FIG. 1B shows one embodiment of a portion of the catalyst mixing chamber 2 where catalyst 4 may be added via inlet 20 and alcohol 5 is introduced via inlet 21. Inlet 21 may be configured to introduce the alcohol component 5 tangentially to the wall of the catalyst mixing chamber 2 to provide a circular mixing flow (turbulent action) with solid catalyst 4. Optionally, mixing balls 5A may be include in the catalyst mixing 2 to enhance dissolution of catalyst 4.

Ratios of dissolved catalyst component 4 to alcohol component 5 used in forming the catalyst solution 3A may range from about 0.02 to about 0.15 parts by weight catalyst 4 for 1 part by weight of alcohol 5. typically, the ratio will be from about 0.05 to about 0.07 parts catalyst 4 for every 1 part of alcohol 5.

Typical reactant charges for producing a batch of alkyl fatty acid ester product using the method of the present invention for use as “biodiesel” are as follows: 1.0-1.5 kilograms of solid catalyst (sodium hydroxide), 8 gallons of alcohol component (methanol), and 40 gallons of triglyceride component.

Transesterification reactions conducted according to the present invention typically involve (a) combining at least one solid catalyst component 4 with an alcohol component 5 in the presence of a turbulent device (for example, see nozzle inlet 21 in FIG. 1B) for wetting the solid catalyst component 4 with the alcohol component 5; (b) forming a catalyst solution 3A comprising transferring a mixture of the solid catalyst component 4 and the alcohol component 5 form a catalyst mixing chamber 2 to a catalyst solution chamber 3 while filtering the mixture of the solid catalyst component 4 and the alcohol component 5 to prevent transfer of the solid catalyst component 4 from the catalyst mixing chamber 2 to the catalyst solution chamber 3; (c) combining the catalyst solution 3A with a triglyceride component 15 in a catalyst-triglyceride mixing chamber 8 comprising; (i) transferring the catalyst solution 3A from the catalyst solution chamber 3 into a first inlet 10 of a turbulent flow suction device 9; and (ii) transferring the triglyceride component 15 into a second inlet 11 of the turbulent flow suction device 9; and (d) producing a mixture 12A of transesterfication reaction products in a holding chamber 13 comprising circulating the triglyceride component 15 from the holding chamber 13 through a triglyceride feed line 15A to the second inlet 11 of the turbulent flow suction device 9, after all of the catalyst solution 3A has been transferred from the catalyst solution chamber 3, for a time sufficient to produce alkyl fatty acid ester product in the holding chamber 13.

Typically, transesterification reactions conducted according to the present invention include (a) terminating circulation of the triglyceride component 15 through the triglyceride feed line 15A; (c) allowing the transesterifiacation reaction products to settle into layers in the holding chamber 13; and (d) separating the alkyl fatty acid ester product form residual triglyceride 15, residual alcohol 5 and glycerol components in the holding chamber 13. Isolation of the alkyl fatty acid ester product id typically conducted by gravity separation of the layers in holding chamber 13, followed by drying of the alkyl fatty acid ester; no distillation operations are required.

Typically, the triglyceride component 15 is circulated through triglyceride feed line 15A for a time sufficient time to produce alkyl fatty acid ester product in the holding chamber 13. Typical contact (circulation) times are form about 30 minutes to about 100 minutes; more typically from about 45 minutes to about 90 minutes. Typical times for transferring all of the catalyst solution 3A formed in catalyst solution chamber 3 are from about 10 to about 30 minutes, more typically from about 15 to about 20 minutes.

Temperature conditions for using the method of the present invention include providing the triglyceride component 15 at a temperature of approximately 120 degrees Fahrenheit (F) prior to charging the triglyceride component 15 to the holding chamber 13, although this temperature may range from about 90 degrees F. to about 130 degrees F. During the reaction stage of transesterification no external heating or cooling is typically required; the temperature within the holding chamber 13 during recirculation of the triglyceride component 15 is typically from about 90 degrees F. to about 130 degrees F.

In the early stages of the transesterification reaction, the mixture 12A exiting outlet 12 forms on top of the triglyceride component 15 in holding chamber 13. As the transesterification progresses, more of the triglyceride component 15 is depleted as it is circulated through triglyceride feed line 15A into catalyst-triglyceride mixing chamber 8. In the latter stages of the transestrifation reaction the composition of the contents of holding chamber 13 comprise alkyl fatty acid ester product, glycerol byproduct, and residual catalyst 4 and alcohol 5 components.

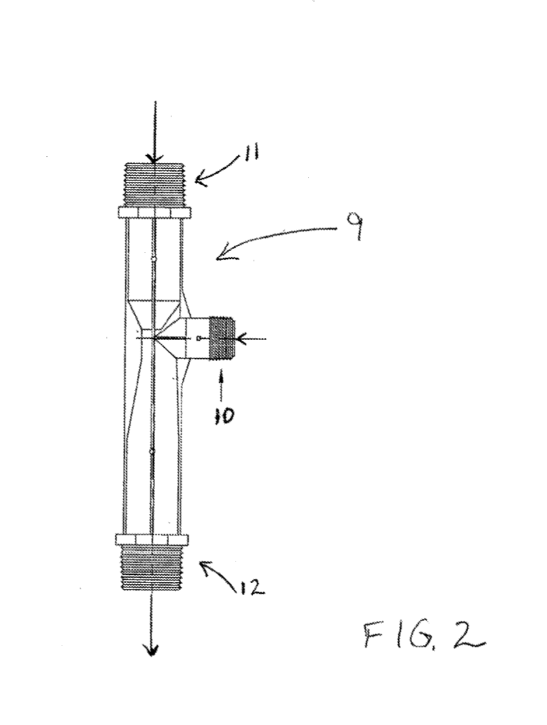

FIG. 2 provides a detailed illustration of one embodiment of a turbulent flow suction device 9 useful in the present invention. Inlet 11 provides for entry of the triglyceride component 15, inlet 10 provides for entry of catalyst solution 3A, and outlet 12 provides for exit of the mixture 12A of catalyst solution 3A and triglyceride component 15 into holding chamber 13. By example, turbulent flow suction device 9 may be a venturi tube being about 222 millimeters in length, with inlet 11 and outlet 12 being 1 inch MNPT fittings, inlet 10 being a¾ inch MNPT fitting. Additional component parts may be positioned between inlet 10 and outlet7 (see FIG. 1A), such as a spring, ball, seat, cap, and 0.5 inch MNPT fitting, to provide appropriate suction during operations of the apparatus.

We claim:

1. A transesterification reaction apparatus comprising:
(A) a catalyst dissolution chamber comprising:
(i) catalyst mixing chamber comprising:
(a) inlets for solid catalyst and alcohol components; and
(b) turbulating device for wetting the solid catalyst component with the alcohol component; and
(ii) catalyst solution chamber coupled to the catalyst mixing chamber comprising:
(a) filtration device located at an interface of the catalyst mixing chamber and the catalyst solution chamber for preventing transmission of solid catalyst from the catalyst mixing chamber to a catalyst solution comprising dissolved catalyst component in the catalyst solution chamber; and
(b) outlet for the catalyst solution;
(B) a catalyst-triglyceride mixing chamber comprising:
(i) turbulent flow suction device;
(ii) first inlet for transferring the catalyst solution into the turbulent flow suction device, wherein the first inlet is coupled to the outlet form the catalyst solution chamber of the catalyst dissolution chamber;
(iii) second inlet for transferring a triglyceride component into the turbulent flow device, wherein the second inlet is coupled to a triglyceride feed line; and
(iv) outlet for transfer of a mixture of the catalyst solution and the triglyceride component;
(C) a holding chamber comprising a container foe holding the triglyceride component and transesterifaction reaction products; and
(D) a fluid transfer device coupled to the holding chamber for transfer of the triglyceride component through the triglyceride feed line to the second inlet of the catalyst-triglyceride mixing chamber.

2. The reaction apparatus of claim 1 wherein the turbulating device comprises a nozzle configured to deliver the alcohol component tangentially to a wall of the catalyst mixing chamber.

3. The reaction apparatus of claim 1 wherein the filtration device comprises a metal screen.

4. The reaction apparatus of claim 1 wherein the turbulent flow suction device comprises a venturi tube.

5. The reaction apparatus of claim 1 wherein the container of the holding chamber encloses the catalyst dissolution chamber and the catalyst-triglyceride mixing chamber in an upper portion of the holding chamber.

6. A method for transesterification comprising, but not all necessary in order shown:
(A) combining at least one solid catalyst component with an alcohol component in the presence of a turbulating device for wetting the solid catalyst component with the alcohol component;
(B) forming a catalyst solution comprising transferring a mixture of the solid catalyst component and the alcohol component from a catalyst mixing chamber to a catalyst solution chamber while filtering the mixture of the solid catalyst component from the catalyst mixing chamber to the catalyst solution chamber;
(C) combining the catalyst solution with a triglyceride component in a catalyst-triglyceride mixing chamber comprising:
(a) transferring the catalyst solution from the catalyst chamber into a first inlet of a turbulent flow suction device; and
(b) transferring the triglyceride component into a second inlet of the turbulent flow suction device; and
(D) producing a mixture of transsesterification reaction products in a holding chamber comprising circulating the triglyceride component from the holding chamber trough a triglyceride feed line to the second inlet of the turbulent flow suction device, after all of the catalyst solution has been transferred from the catalyst solution chamber, for a time sufficient to produce alkyl fatty acid ester product in the holding chamber.

7. The method of claim 6 comprising circulating the triglyceride component in step (D) for from about 30 minutes to about 100 minutes.

8. The method of claim 6 comprising circulating the triglyceride component in step (D) for from about 45 minutes to about 90 minutes.

9. The method of claim 6 comprising transferring all of the catalyst solution from the catalyst solution chamber in from about 10 minutes to about 30 minutes.

10. The method of claim 6 comprising transferring all of the catalyst solution from the catalyst solution chamber in from about 15 minutes to about 20 minutes.

11. The method of claim 6 further comprising:
(A) terminating circulation of the triglyceride component through the triglyceride feed line;
(B) allowing the transesterification reaction products to settle into layers in the holding chamber; and
(C) separating the alkyl fatty acid ester product from residual triglyceride, residual alcohol and glycerol components in the holding chamber.

12. The method of claim 6 further comprising combining the catalyst solution with the triglyceride component in the catalyst-triglyceride mixing chamber in a ratio of from about 1/3 to about 1/10 by volume by adjusting relative flow rates of the catalyst solution and triglyceride component into the first and second inlets, respectively, of the turbulent flow suction device.

13. The method of claim 12 comprising providing the ratio of catalyst solution to triglyceride component of from about 1/4 to about 1/6 by volume.

14. The method of claim 6 further comprising forming the catalyst solution in a ratio of about 0.02 to about 0.15 parts by weight of catalyst component per 1 part by weight of alcohol component.

15. The method of claim 14 comprising providing the ratio of from about 0.05 to about 0.07 parts by weight of catalyst component per 1 part by weight of alcohol component.

16. The method of claim 6 further comprising selecting the solid catalyst component from one of more of the group consisting of lithium hydroxide, sodium hydroxide and potassium hydroxide.

17. The method of claim 6 further comprising selecting the alcohol component from one or more of the group consisting of methanol, ethanol, n-propanol and isopropanol.

18. The method of claim 6 further comprising selecting the triglyceride component from one or more of the group consisting of triglycerides based on esters of (C₈-C₂₄)fatty acids.

19. The method of claim 18 comprising selecting the (C₈-C₂₄) fatty acids from one or more of caprylic, lauric, myristic, palmitic, stearic, arachnic, behinc, lignoceric, oleic, linoleic, linolenic and erucid acids.

20. The method of claim 6 comprising the mixture of transesterification reaction products comprising an alkyl fatty acid ester and glycerol.

21. The method of claim 20 wherein the alkyl fatty acid ester comprises one or more of methyl, ethyl and propyl esters.
